(19) United States
(12) Patent Application Publication
(10) Pub. No.: US 2021/0196585 A1
(43) Pub. Date: Jul. 1, 2021

(54) BIODEGRADABLE MICROBEADS

(71) Applicant: Corning Incorporated, Corning, NY (US)

(72) Inventors: Theresa Chang, Painted Post, NY (US); Ye Fang, Painted Post, NY (US); David Henry, Fontaine le Port (FR); Wendell Porter Weeks, Corning, NY (US); Ying Wei, Painted Post, NY (US)

(21) Appl. No.: 17/199,950

(22) Filed: Mar. 12, 2021

Related U.S. Application Data
(62) Division of application No. 15/603,098, filed on May 23, 2017.
(60) Provisional application No. 62/342,391, filed on May 27, 2016.

(51) Int. Cl. A61K 8/11; A61K 8/73; A61Q 19/10; A61K 8/02; A61K 8/19; A61K 8/25; A61K 8/26

(52) U.S. Cl. CPC A61K 8/11; A61K 8/73; A61K 8/733; A61Q 19/10; A61K 8/0241; A61K 8/19; A61K 2800/413; A61K 8/26; A61K 2800/28; A61K 2800/651; A61K 2800/624; A61K 2800/412; A61K 8/25

(57) ABSTRACT

An exfoliant composition including:
a microbead comprising a core and a shell:
the core comprising an abrasive particle having an average particle size of from 50 to 1,000 microns; and
the shell comprising a hydrogel. Also disclosed is a method of making the exfoliant composition and a method of using the exfoliant composition.

This application claims the benefit of priority under 35 U.S.C. § 119 of U.S. Provisional Application Ser. No. 62/342,391 filed on May 27, 2016 the content of which is relied upon and incorporated herein by reference in its entirety.

### CROSS REFERENCE TO RELATED APPLICATIONS

This application is related to commonly owned and assigned copending patent applications: U.S. Provisional Application Ser. No. 61/838,452 filed on Jun. 24, 2013, now U.S. Ser. No. 14/899,394, entitled “CELL CULTURE ARTICLE AND METHODS THEREOF”; the contents of which are relied upon and incorporated herein by reference in their entirety, but does not claim priority thereto.

This application is also related commonly owned and assigned USSN Provisional Application Nos., filed concurrently herewith:

62/342,384, entitled “BIOACTIVE ALUMINOBORATE GLASSES”;

62/342,377, entitled “MAGNETIZABLE GLASS CERAMIC COMPOSITION AND METHODS THEREOF”;

62/342,381, entitled “LITHIUM DISILICATE GLASS-CERAMIC COMPOSITIONS AND METHODS THEREOF”;

62/342,411, entitled “BIOACTIVE GLASS MICROSPHERES”; and

62/342,426, entitled “BIOACTIVE BOROPHOSPHATE GLASSES”; but does not claim priority thereto.

The entire disclosure of each publication or patent document mentioned herein is incorporated by reference.

### BACKGROUND

The disclosure relates to biodegradable microbeads and methods thereof.

### SUMMARY

In embodiments, the disclosure provides a microbead composition comprising an abrasive core particle and a hydrogel shell.

In embodiments, the disclosure provides an exfoliant composition

In embodiments, the disclosure provides an exfoliant composition comprising: a microbead comprising a core and a shell, the core comprising an abrasive particle having an average particle size of from 50 to 5,000 microns; and the shell comprising a hydrogel.

In embodiments, the disclosure provides a microbead composition comprising an abrasive core particle, such as a water insoluble calcium salt, sand, a bio-glass, or calcium glass, and a hydrogel shell such as a ionically cross-linked, polygalacturonic acid (PGA).

In embodiments, the disclosure provides a microbead composition having a crosslinked hydrogel shell, such as crosslinked with a polyvalent cation, to form the microbead having controllable size, and controllable moduli associated with the abrasion properties of the microbeads.

In embodiments, the disclosure provides methods of making and methods of using the microbead or exfoliant composition.

### BRIEF DESCRIPTION OF THE DRAWINGS

In embodiments of the disclosure:

FIGS. 1A and 1B shows [PRIOR ART] polygalacturonic acid (PGA)(1A) (a.k.a. pectic acid); and PGA crosslinked by a calcium ion (1B).

FIGS. 2A and 2B show example microbead production methods by: PGA droplet addition to a calcium chloride solution (2A); and calcium carbonate crystallite addition to a PGA solution (2B).

FIGS. 3A to 3D show light microscope images of stages of microbead preparation.

FIG. 4 shows exemplary composite microbeads after drying, i.e., having a PGA shell bound to a CaCO₃ core particle.

FIG. 5 shows exemplary composite microbeads after rehydration in deionized water, i.e., having a hydrogel of a re-hydrated PGA shell bound to a CaCO₃ core particle.

FIG. 6 shows accelerated dissolution kinetics of Ca²⁺ ion release from exemplary core material pairs as a function of pH 4 (left bar) and pH 6 (right bar) after 24 hrs in acetate buffer.

FIG. 7 shows an image of isolated core-shell microbeads having an aluminoborate glass core and a PGA shell that is cross-linked with Ca²⁺ ions.

FIG. 8 shows an image of isolated core-shell microbeads having a precipitated calcium carbonate core and a PGA shell that is cross-linked with Ca²⁺ ions.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

In embodiments, the disclosed compositions, methods of making, and methods of using provide one or more advantageous features or aspects, including for example as discussed below. Features or aspects recited in any of the claims are generally applicable to all facets of the invention. Any recited single or multiple feature or aspect in any one claim can be combined or permuted with any other recited feature or aspect in any other claim or claims.

### Definitions

“Hydrogel,” “hydrogel-shell,” “hydrogel layer,” “gel layer,” “gel source,” or like terms refer to at least one polymeric material that can form a shell on a core particle; is capable of absorbing large quantities of fluids such as water; and is further capable of retaining the absorbed fluids. The particle hydrogel or particle gel is distinct from a formulation “gel” mentioned below.

In embodiments, the hydrogel material can comprise, for example, a polymer of a carboxylic acid or a derivative thereof, such as acrylic acid, PGA, alginate, or mixtures thereof. In embodiments, the carboxylic acid containing polymer preferably can be naturally occurring polymers such as PGA or alginate. These polymers are rendered water-insoluble or less water soluble by cross-linking the carboxyl group-containing polymer chains using conventional cross-linking agents such as a divalent or a polyvalent cation. The degree of cross-linking in hydrogel and hydrogel-forming materials determines their water-solubility and can also be an important factor in establishing two other characteristics of fluid absorbing hydrogels, i.e., absorbent capacity and gel strength. Absorbent capacity of “gel volume” is a measure of the amount of water or fluid which a given amount of hydrogel-forming material will absorb. Gel strength relates to the tendency of the hydrogel formed from such material to deform under an applied stress such as an exfoliation or abrasion condition.

“Gel,” “formulation gel,” or like terms refer to a viscous formulation or carrier commonly used in cosmetics and in personal care products. Cosmetic and skin care products can include multiple ingredients of different physicochemical properties. A formulation balances all of the ingredients in a particular product. Each ingredient has its own specific physicochemical parameters including solubility/miscibility, melting point, specific gravity, viscosity, etc. Typically, the formulation of any cosmetic product is about structural and functional considerations. There must be a strong affinity among the structural ingredients to produce a desired physical form, and which form has the proper function. The “formulation gel” is distinct from the above mentioned hydrogel.

“Carrier” or like terms refer to a formulation that the microbeads are suspended in, such as a liquid, a soap, a soap solution, a gel, a cream, a lotion, a powder, and like formulation suspension media, or a mixture thereof.

“PGA,” or like terms can refer to any polygalacturonic acid, such as a pectic acid, a partially esterified pectic acid having a degree of esterification, for example, from 1 to 50 mol %, or mixtures thereof, or salts thereof.

“X-PGA,” or like terms can refer to any crosslinked polygalacturonic acid, such as a pectic acid, a partially esterified pectic acid having a degree of esterification of, for example, from 1 to 50 mol %, or mixtures thereof, or salts thereof, that further includes any covalent cross-linking, ionic cross-linking, or combinations of covalent and ionic cross-linking.

“Particle,” “microparticle,” “bead,” “microbead,” “microbead,” hollow bead,” “hollow microbead,” “hollow microparticle,” or like terms refer to a solid matter which has a regular (e.g., spherical, or ovoid) or irregular shape. Specifically, microbeads widely used in cosmetics as exfoliating agents and in personal care products such as toothpaste are defined as manufactured particles of less than five millimeters in their largest dimension (see C. Copeland: Microbeads: An Emerging Water Quality Issue, fas.org, Jul. 20, 2015). Microbeads are commercially available in particle sizes from 10 micrometers to 1 millimeter. Hollow microbead refers to microbeads having a hollow structure, wherein the microbead has, for example, an empty center or empty core, which can be filled with air or other gases, surrounded by the solid matter (e.g., glass or ceramics). A biodegradable microbead refers to a microbead that is stable in typical formulations commonly used in cosmetics and personal care products, but will degrade in time when exposed to an ambient environment outside the formulation.

“Glass,” “glasses,” or like terms can refer to a glass or a glass-ceramic.

“Glass article,” or like terms can refer to any object made wholly or partly of glass or a glass-ceramic.

“Include,” “includes,” or like terms means encompassing but not limited to, that is, inclusive and not exclusive.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, times, and like aspects, and ranges thereof, are for illustration only; they do not exclude other defined values or other values within defined ranges. The composition and methods of the disclosure can include any value or any combination of the values, specific values, more specific values, and preferred values described herein, including explicit or implicit intermediate values and ranges.

Many cosmetic and personal grooming products contain microbeads as gentle abrasives. Among other functions, the primary purpose of these microbeads is to promote exfoliation of dead skin through mild abrasive action. As these products must be suitable for a wide range of skin types, it is important for the abrasive to have the proper level of abrasiveness. Too little abrasiveness and the product is ineffective. Too much abrasiveness and the product may damage the user's skin. For this reason many products on the market are made from polymers, such as polyethylene. Plastic beads can easily and cheaply be made to have the correct size distribution, shape, and hardness, which in turn leads to the correct level of abrasiveness. However, a major drawback of many of these polymer beads is that they are not practically biodegradable.

Polyethylene is a common choice for a bead material found in personal care products today. Depending on the disposal environment, degradation of polyethylene can take from years to centuries to degrade. Many cosmetic and personal grooming products end up being disposed of into a sewage system and eventually may reach an ocean. Furthermore, non-bio-derived polymers tend to have unsafe intermediate degradation products, so that even if they do degrade they are still potentially harmful to wildlife and can even enter the food-chain by bioaccumulation in food animals, such as fish. Even plastics designated as biodegradable such as ones utilized for tissue engineering (such as poly ε-caprolactone (PCL) and poly lactic acid (PLA)) can be harmful to oceanic life as the degradation rate is very different in an oceanic environment vs. in tissue culture or more in vivo-like environments.

One solution to this problem is to use bio-derived polymers as the source for the beads. However, one must choose a material that has the correct bio-degradation rate. If the bio-degradation is too fast, the stability of the beads and the shelf-life of a product containing the beads may be jeopardized. One must also be able to produce the beads in the correct size and size distribution and be able to disperse the beads into aqueous media. Many bead containing products are formulated in aqueous media, such as an emulsion for creams, or an oil base. The present disclosure addresses many of these issues and provides a biodegradable bead composition of appropriate size, size distribution, hardness, and biodegradation rate.

### PGA Polymers

The microbeads of the present disclosure can be made of at least one ionotropically cross-linked polysaccharide selected from, for example, pectic acid, also known as polygalacturonic acid (PGA), or a salt thereof, or partly esterified pectic acid (PE PGA) known as pectinic acid, or a salt thereof. When pectinic acid is selected, the degree of esterification is preferably less than about 40 mol % since a higher degree of esterification makes bead formation by ionotropic crosslinking ineffective. Without being bound by theory it is believed that a minimum amount of free carboxylic acid groups may be called for to obtain an acceptable level of ionotropic crosslinking.

Polygalacturonic acid (PGA) (FIG. 1A), also known as pectic acid, is an oxidized polysaccharide. It is derived from fruit and some vegetables and is water-soluble. PGA has the useful property of being able to be crosslinked when exposed to divalent or polyvalent cations, such as magnesium, calcium (FIG. 1B), aluminum, or a polycation such as chitosan.

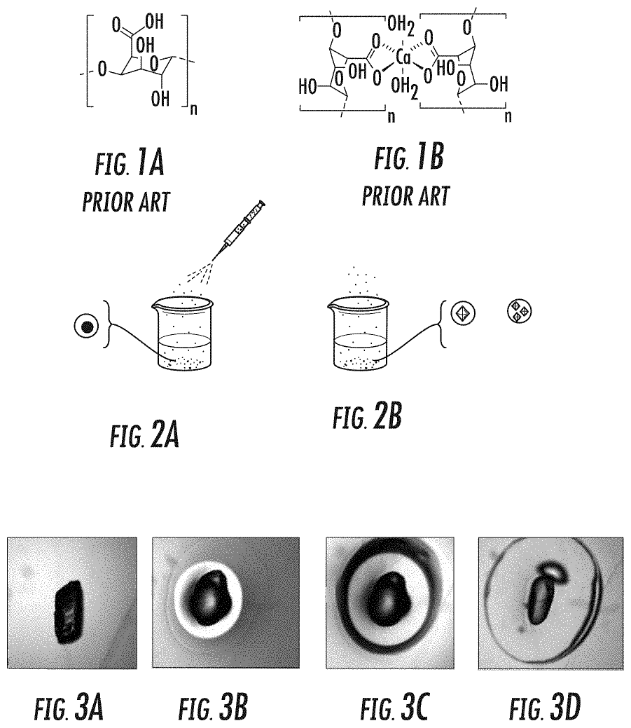

FIGS. 2A and 2B, respectively, show exemplary microbead production methods by: adding a gel source (e.g., PGA) droplets to a gel source crosslinker (e.g., calcium chloride) solution (2A); or adding a gel source crosslinker (e.g., calcium carbonate crystallites) to a gel source solution (e.g., PGA) (2B).

Microbeads can be produced with an outside-in approach (e.g., FIG. 2A) where, for example, a solution of a gel source (e.g., PGA) containing core particles is dispensed as droplets into a solution containing a divalent cation (such as Ca²⁺). The Ca²⁺ crosslinks the exterior of the gel source droplet first from direct contact, then the ions diffuse into the droplet to crosslink the remainder of the gel source droplet to produce microbeads having a crosslinked shell (e.g., hydrogel) encapsulating the core particle(s). In embodiments, the present disclosure provides core-shell microbeads and methods of making the core-shell microbeads, where for example, a core substance is present, suspended, or dispersed in the solution of the cross-linking agent such as a divalent cation (e.g., Ca²⁺). In embodiments, the core substance can include or be coated with a cross-linking agent for the gel source.

Alternatively, the disclosed microbeads can be produced by an inside-out approach (e.g., FIG. 2B), where a solid crosslinker source containing divalent ions such as Ca²⁺ (e.g., single core particles, twins, and like small aggregates or clusters of core particles) for the gel source, is introduced into a gel source solution or shell source solution (e.g., PGA). The gel source solution is preferably acidic (with or without another acid catalyst present), and the gel source crosslinker ions (e.g., calcium ions from CaCO₃) are released from the exterior of the core particles from partial dissolution. The released gel source crosslinker ions (e.g., Ca²⁺) crosslink the gel source material that surrounds the core particle(s), producing a microbead having a crosslinked hydrogel shell situated on and encapsulating one or more of the core particles. In embodiments, the size microbead or the thickness of the crosslinked hydrogel shell can limit diffusion of calcium ions from the core, and limit diffusion of calcium ions through the crosslinked hydrogel shell.

HU200302501A2 entitled “Metal complex of polygalacturonic acid and its production” mentions metal complexes of polygalacturonic acid which also contains M-X-M′ structural unit, and their manufacture. M and M′ stand for identical or different essential metals or trace elements, preferably with a valency of one, two or three, metals in Groups I-III of the Periodic Table, or transitional metals, especially and preferably iron, zinc, magnesium, copper, chromium, molybdenum, cobalt, nickel, manganese, lithium, sodium, potassium or calcium, using one or in some cases more of these. X is selected from —O—, —O(H)—, O(R′)-(R′-polygalacturonate-chain containing a coordinated metal-ion, or another polygalacturonate-chain containing a coordinated metal-ion, or another polygalacturonate-chain C(═O)—, or the residue of the CH— group carrying alcohol OH of galacturonate units), halide ion, hydroxide ion or other, biologically acceptable mono-, or bidendate anion, preferably sulphate, carbonate, hydrogen-carbonate, acetate, lactate, malate, citrate, tartarate.

International Journal of Pharmaceutics 308 (2006), 25-32, mentions an immersion coating methodology using a calcium containing core, immersing the core particles into pectin, then crosslinking the coated pectin with, for example, either calcium or chitosan, using the crosslinking to control release rate of a model drug compound.

U.S. Pat. No. 7,597,900, entitled “Tissue Abrasives,” to Schott, mentions abrasive compositions which include bioactive materials, such as bioactive glass and bioactive ceramics, which provide biological properties such as anti-inflammatory, anti-microbial, anti-oxidant effects, improved wound healing, and/or other beneficial.

In embodiments, the disclosure provides an exfoliant composition comprising:

a microbead comprising a core and a shell: the core comprising an abrasive particle having an average particle size of from 50 to 5,000 microns; and the shell comprising a hydrogel.

In embodiments, the abrasive particle can be, for example, selected from at least one of: CaCO₃, a calcium mineral, a glass, a calcium containing glass (e.g., a calcium containing silicate, borate, borophosphate, and like glasses), a silica, a porous silica doped with calcium, a sand such as a silica sand or a carbonate sand, or a mixture thereof.

In embodiments, a glass abrasive particle, either doped or undoped with a divalent cation, was demonstrated as a useful abrasive. Glasses that leach Ca²⁺ and glasses that do not leach Ca²⁺ have been demonstrated as useful abrasives.

In embodiments, the hydrogel can be selected, for example, from at least one of: a cross-linked polygalacturonic acid (X-PGA); cross-linked alginate; and like materials, or a mixture thereof (PGA or alginate was, for example, crosslinked upon the addition of a core ingredient, and the cross-linking was, for example, completed in the soaking step using CaCl₂).

In embodiments, the cross-linked polygalacturonic acid can be, for example, cross-linked with a polyvalent cation.

In embodiments, the polyvalent cation can be selected, for example, from at least one of: Ca²⁺, Cu²⁺, Al³⁺, Fe³⁺, Mg²⁺, Pb²⁺, Cd²⁺, Ba²⁺, Sr²⁺, Co²⁺, Ni²⁺, Zn²⁺, Mn²⁺, chitosan, or mixtures thereof. Preferably, the cation is environmentally innocuous. Chitosan is a linear aminated polysaccharide composed of randomly distributed β-(1-4)-linked D-glucosamine (deacetylated unit) and N-acetyl-D-glucosamine (acetylated unit). While magnesium ion (Mg²⁺) forms hydrogels with alginate it requires a longer gel time and a considerably higher concentration of the source of magnesium ion compared to calcium ion hydrogels (see Soft Matter, 2012, 8, 4877).)

In embodiments, the shell can modulate (i.e., attenuates or mitigates) the abrasiveness of the abrasive particle.

In embodiments, the type and extent of the crosslinking of the cross-linked polygalacturonic acid can control the environmental biodegradability of the microbead.

In embodiments, the microbead (i.e., suspended in a formulation) can have a durable shell, where the shell durability can be measured by calcium ion release from the core of a core-shell microbead. If the shell separates from the core particle in a formulation used in a cosmetic or personal care product, it means the microbead can have an unsatisfactory shelf-life or shelf-stability, and the shell may be unsatisfactory as a shell material for use in personal care products.

In embodiments, the microbead formulation (i.e., the microbead suspended in a formulation) has a biodegradability that is greater than comparable plastic microbeads formulations. The biodegradability of microbeads can depend on their structure, type, and physiochemical properties. In embodiments, the naturally occurring polymer-based shell will degrade by microbial action (e.g., bacteria) or agents (e.g., acid, base, corrosive materials, etc.) presented in environment (e.g., sea water, sewage), and the core material (e.g., bioactive glass, or CaCO₃ minerals) will dissolve over time. In embodiments, the naturally occurring polymer-based shell of the present disclosure will dissociate from the core material by releasing or loosing crosslinking cations after entering the ambient environment (e.g., sea water, sewage system, etc.), so the core material can be dissolved (e.g., bioactive glass, CaCO₃ minerals) or released to environment (e.g., sand). Nonetheless, compared to typical plastic microbeads, which can often last over decades or centuries, the disclosed biodegradable microbeads have a substantially shorter half-life in ambient environments. In ambient environments, the disclosed biodegradable microbeads can have a half-life as short as one month and can overcome the issue associated with typical plastic microbeads, which can pose an environmental hazard when disposed of in waste water. Because the microbeads pass through sewage treatment plants without being filtered out, their disposal has resulted in plastic particle water pollution with microplastics (see Fendall, L. S., et al., “Contributing to marine pollution by washing your face: microplastics in facial cleansers”. Marine Pollution Bulletin 58 (8): 1225-1228 (2009)).

In embodiments, the disclosed microbead composition can have a shell that is durable and can resist acid degradation, base degradation, or both.

In embodiments, the disclosed microbead compositions can have an environmental biodegradability of from 1 week to 5 years, which is significantly less than plastic microbeads.

In embodiments, the disclosed composition can further comprise, for example, a carrier. In embodiments, the carrier can be selected, for example, from a liquid, a soap, a soap solution, a gel, a cream, a lotion, a powder, or a mixture thereof.

In embodiments, the microbead can have a high elastic modulus core and a low elastic modulus shell where the core elastic modulus is of from 50 to 90 GPa, and the shell elastic modulus is of from 100 to 500 kPa (i.e., that is less than about 1 MPa). The elastic modulus of the disclosed microbead can be, for example, greater than polyethylene and less than silica sand.

In embodiments, the disclosure provides a method of making the above mentioned exfoliant composition comprising:

forming a hydrophilic shell, i.e., hydrogel, on the surface of an abrasive core particle to form a microbead.

In embodiments, the method of making can further comprise, sequentially or simultaneously, cross-linking the hydrophilic shell on the surface of the abrasive core particle.

In embodiments, the cross-linking can be accomplished, for example, with a polyvalent cation and an acid catalyst, such as acetic acid.

In embodiments, the disclosure provides a method of using the above mentioned exfoliant composition comprising:

contacting a skin surface with the exfoliant composition.

In embodiments, the method of using can further comprise mechanically working the exfoliant composition onto or into the skin surface.

In embodiments, the method of using can further comprise removing the mechanically worked exfoliant composition from the skin surface, e.g., by washing with water or by wiping off.

Aspects of the present disclosure are advantaged is several respects, including, for example, the disclosed microbeads and their formulations provide: biodegradability; particle size control; abrasiveness control, modulus control; and a core-shell system for encapsulating active or inactive ingredients in the microbead core.

### General Preparative Procedures—Method of Making Spherical Hydrogel Particles

Crosslinking phenomenon can be used to form spherical gel particles (microbeads) of, for example, PGA by exposing the PGA to the proper concentration of crosslinking ions.

In embodiments, the bead can include at least one inorganic abrasive filler as the core particle. The inorganic abrasive filler core particle is advantageously a water-insoluble salt of a divalent cation, preferably calcium, e.g., calcium carbonate, calcium minerals such as calcite and aragonite. In addition to the inorganic abrasive filler's role as an abrasive, one example, calcium carbonate, can also serve the role of a calcium ion source leading to internal ionotropic gelation of a polysaccharide binder resulting in an enhanced bead crosslinking.

There are at least two ways of making PGA gel microbeads. The first approach, referred to as the “outside-in” approach, produces microbeads by dispensing droplets of a solution of PGA (e.g., atomization) with or without suspended abrasive fillers into a solution containing polyvalent cations, such as calcium chloride (Ca²⁺) or copper sulfate (Cu²⁺). Upon exposure to the cations in the solution, the PGA droplets begin to crosslink from the outside inward. As the cations diffuse into the droplets they become more and more crosslinked, until the entire droplet is made of crosslinked PGA. The size of the PGA solution droplets will control the size of the microbeads produced. This initial concentration of PGA (among other factors such as molecular weight) in the droplets will control the hardness of the microbeads. Low concentrations will lead to a more open network of lower density, while higher concentrations will lead to a denser, tighter network.

The crosslinking effectiveness of different divalent cations for either alginate or for PGA is generally known, for example, with respect to the relative binding strength or gel strength v. cation type. Alginate's affinity toward the different divalent ions (2+) has been shown to decrease in the order: Pb>Cu>Cd>Ba>Sr>Ca>Co, Ni, Zn>Mn (see Y. A. Mørch, et al., Effect of Ca²⁺, Ba²⁺, and Sr²⁺on Alginate Microbeads, Biomacromolecules, 2006, 7, 1471-1480). With respect to the present disclosure some of these divalent ions would be contraindicated because of, for example, toxicity. Although not limited by theory it is believed that the calcium ions crosslink the alginate or the PGA through their carboxylic acid sites. An orthosilicate ester, such as a tetraethyl orthosilicate (TEOS), can crosslink the alginate or the PGA through their hydroxyl groups. The TEOS can increase the hydrogel crosslinking and increase the durability of the microbeads in, in for example, soap or water suspensions.

In embodiments, the disclosure provides a method of making a hydrogel coated microbead having at least a cross-linked hydrogel shell comprising, for example: dispensing a solution (e.g., atomization) of PGA in a solution containing polyvalent cations (e.g., Ca²⁺ or Zn²⁺, or a polycation such as chitosan), creating a shell of crosslinked material starting from the outer sphere of the PGA droplet (i.e., external gelation). Alternatively, the PGA gel beads could be produced by introducing soluble salts of divalent (such as Ca²⁺, or trivalent such as Fe³⁺, etc.) into a solution of PGA, allowing the crosslinking to occur from the inside out (i.e., internal gelation). The use of soluble salts may be challenging since the gelation may begin immediately after introduction of the multivalent ions, without some method of controlling gelation speed, such as temperature or other process levers. Use of a mostly insoluble (sparingly/partially soluble) polyvalent salt (di, tri, or multivalent) as nanoparticles to partially crosslink PGA to form higher viscosity, to allow finer control of particle size by control of solution viscosity. The rate of dissolution (release) of the ions and the extent of crosslinking can be finely controlled by addition of acids (to aid solubility) or potentially a photoacid to control timing of gelation. This partially crosslinked material can then be further crosslinked from the exterior by dispensing into a solution of the soluble salt or an acidic solution to release the calcium ions near the surface of the bead. This last step also can control the extent of microbead crosslinking by ion concentration and time of contact with the hardening (soluble divalent salt) solution (i.e., both internal and external gelation).

Use of a sparingly soluble multivalent salt can seed hydro-gel formation around the particle(s). Aluminum salts or copper salts may not be preferable for cosmetics but can be a demonstrative example.

The microbeads can also have other inorganic or organic fillers incorporated into them during the bead growth phase, through pre-suspension in the PGA solution, to alter the average hardness of the beads to render them suitable for different applications.

The microbeads can be post processed to further crosslink if necessary (mechanical, e.g., can potentially be crosslinked by gamma irradiation), or to impart functional or aesthetic properties such as including a colorant.

The hydrogel shell or gel layer in any of the preparative examples can also be used to introduce active ingredients as needed for the specific product.

### Alternative Method of Making Spherical Hydrogel Particles

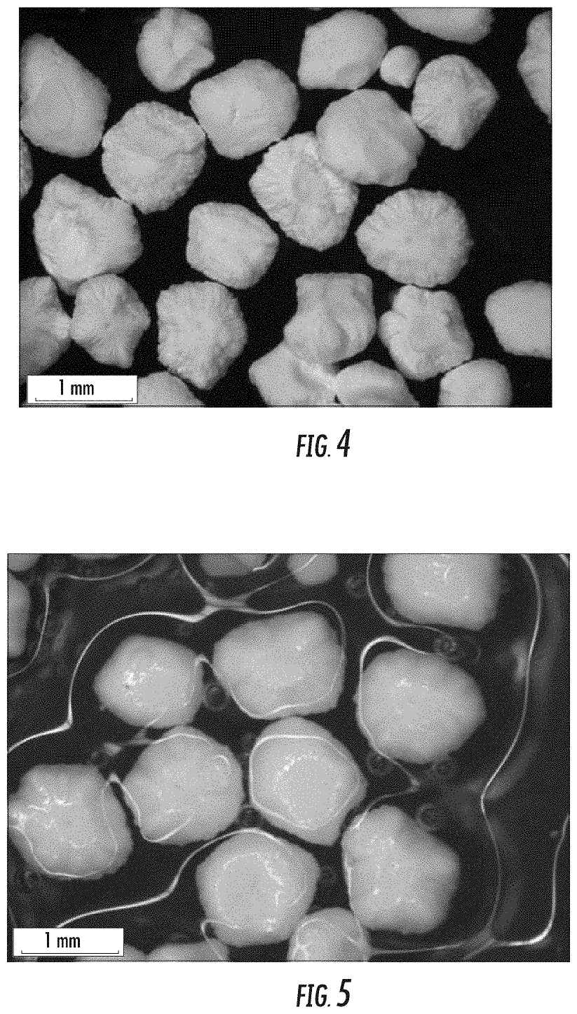

A second approach, referred to as the “inside-out” approach, produces microbeads by introducing the cations to a solution of the PGA as tiny crystallites (or agglomerates). Gradual dissolution of the ions causes the PGA to crosslink in proximity to the source of those ions and to form a gel layer around the crystallites. This leads to growth of microbeads from the inside out. The size of the microbeads is controlled by the size of the crystallites as well as concentration of the PGA solution and by how long the beads are allowed to grow. When the beads reach the desired size, they may be filtered from the solution and washed. This can be done, for example, using soluble salts such as copper sulfate as shown in FIGS. 3B and 3C, or sparingly soluble salts (water insoluble) such as calcium carbonate. FIGS. 3A to 3D show light microscope images of stages of microbead preparation. FIG. 3A is a core particle produced by seeding copper sulfate crystallite particles (3A) into a PGA solution. FIG. 3B is a microbead having a copper sulfate crystallite core and a PGA encapsulating shell (white surround). The dark outer ring is an image artifact resulting from light interference. FIG. 3C is a microbead having a copper sulfate crystallite core and a cross-linked PGA hydrogel shell. The dark outer ring is believed to be an imaging artifact. FIG. 3D is a microbead of FIG. 3C that has been washed with water that shows a distinct crystallite core and a cross-linked PGA hydrogel shell.

A combination of both the outside-in and inside-out microbead preparative methods can also provide a pre-gelation method where, for example, nanocrystallites are introduced into a solution of PGA to form crosslinks that do not fully crosslink the gel, but instead increase the viscosity of the resulting partially crosslinked PGA solution. The droplets can then be dispensed from this higher viscosity solution into a solution containing soluble divalent (or higher) cations. This combined method results in control of viscosity, resulting in a more uniformly gelled microbead structure (with or without a core particle).

The combined method has the advantage of providing a supply of excess cations within each gelled PGA microbead. This can accelerate the production of shell-core microbeads, but can also possibly reduce the degradation of the microbeads with time if they are placed in a solution of low ionic strength, such as pure water. For the microbeads produced using the outside-in crosslinking approach, the PGA shell will dissociate and degrade over time when placed in water due to the diffusion of finite cations out of the shell layer. However, microbeads made with internal crystallites have a built-in reserve of cations that are able to replace any cations that are lost through diffusion. This provides them with enhanced lifetime and a wider array of environments in which they may be useful. For the microbeads produced using the combined approach, there are the excess cations, which will slow down the diffusion of cations out of the microbeads, thus slowing down the dissociation of crosslinked PGA molecules, when the microbeads are placed in low ionic strength solutions.

Adding a large excess of micronized insoluble CaCO₃ particles (i.e., more Ca ion than is required for ionotropic crosslinking only) dispersed in the PGA solution can be a straight forward way to tailor mechanical properties and abrasiveness of the resulting beads. Such PGA organic shell and CaCO₃ inorganic core composite droplets can be crosslinked by external (outside-in) gelation, internal (inside-out) gelation, or a combination of both methods, as described above. Control of the initial pH of the PGA/CaCO₃ can be utilized to adjust solution life time to prevent premature gelation for processing. In embodiments, a microbead having one-core and one-shell structure is preferred. In embodiments, a microbead having a plurality of cores surrounded by single shell structure can be prepared.

It has unexpectedly been discovered that even submicron size inorganic fillers can provide an abrasive effect when they are included as a filler in at least one of the disclosed composite microbead particles.

As an example, Albafil PCC powder from Mineral Technologies, Inc., with an average particle size of 0.7 microns is particularly suited to practice the invention. Smaller particle size PPC might be used but usually a too fast gelation occurs which makes the bead formation process difficult.

In embodiments, other inorganic fillers can be added to the composition such as various forms of silicon dioxide, aluminum oxide, magnesium oxide, titanium oxide, aluminosilicate, silicon carbide, powdery silica, marble, dolomites, aragonites, feldspars, gypsum, clays, kaolins, and like fillers.

Optionally, organic fillers can be also added provided that they are biodegradable.

Preferably the composite beads are formed or shaped by extruding or “spraying” the polysaccharide/inorganic abrasive filler, i.e. CaCO₃, suspension into a hardening bath made of a solution containing an appropriate amount of multivalent cations.

### Higher Modulus Particles Having a Thin Gel Layer

Other shapes may also be achievable through use of water insoluble divalent, etc., salt particles (although soluble in acidic medium) where local dissolution is achieved by interaction of divalent ions with the carboxylic acids of the PGA. In this scenario, the gel layer surrounding the salt particle will be relatively “thin” where the gel layer thickness is controlled by solubility of the salt in the presence of the PGA, the pH of the solution, and diffusion of ions not only through a solution of PGA, but through the crosslinked gel, which was observed to be significantly slower. In this case, the modulus of the resulting “bead” will be more significantly influenced by the salt rather than the gel layer. In embodiments, the microbeads can also have other inorganic materials incorporated into them, for example, silica particles, during the bead growth phase, through pre-suspension in the PGA solution, to alter the abrasion properties of the beads and to render them suitable for different applications.

### Modulus Control of Microbeads

Conversely, if a product require a lower modulus bead, it is also feasible to create a lower modulus microbead by use of a slower diffusing cation (i.e. stronger crosslinker such as trivalent ions including Fe³⁺). Dispensing (such as atomization) a PGA solution into an Fe³⁺ containing solution will form a gel layer at the surface of the PGA droplet. However, the diffusion rate of Fe³⁺ is slow through the strongly crosslinked skin, resulting in a gel that is not crosslinked in the center. However, if this system is allowed enough time for the trivalent cation to diffuse to the center of the droplet, the resulting hydrogel will have higher modulus than one created with a divalent ion, for example.

Other methods can be used to further control bead properties such as using a cellulose or a modified cellulose, either dissolved or suspended in a PGA solution. These cellulose and PGA mixtures can be used to, for example, control viscosity, impart functionality, or impart hydrophobicity.

In embodiments, the amount of calcium carbonate can preferably be, for example, of from 1 to 40 wt %, but the amount can be higher if desired. The largest amount of the inorganic filler to be added can be limited by the viscosity of the resulting suspension. The smallest amount of the inorganic filler can be limited by the abrasive properties expected.

The drying step performed at the end of the process can be significant to provide a desired abrasive effect. The ability to rehydrate more or less impacts such abrasive effect.

The ratio of inorganic abrasive filler to hydrogel allows for controlling, for example, the stiffness, compressive strength, and hardness of the composite bead in their rehydrated state.

### Aluminoborate Glasses

In embodiments, the alumino-borate glass composition can comprise a source of, for example:

30 to 65% B₂O₃,

1 to 30% Al₂O₃,

1 to 5% P₂O₅,

3 to 30% Na₂O, and

5 to 30% CaO, based on a 100 mol % total of the composition.

In embodiments, the alumino-borate glass composition can further comprise a source of, for example:

0.1 to 15% K₂O,

0.1 to 15% MgO,

0.1 to 10% SrO, and

0.1 to 5% SO₃, based on a 100 mol % total of the composition.

In embodiments, a more preferred composition can include a source of, for example:

50 to 60% B₂O₃,

2 to 10% Al₂O₃,

1 to 3% P₂O₅,

4 to 10% Na₂O,

6 to 10% K₂O,

6 to 10% MgO, and

20 to 30% CaO, based on a 100 mol % total of the composition.

In embodiments, a most preferred composition can include a source of, for example:

50 to 60% B₂O₃,

5 to 10% Al₂O₃,

1 to 3% P₂O₅,

4 to 8% Na₂O,

6 to 10% K₂O,

6 to 10% MgO,

20 to 24% CaO, and

0.2 to 2% SO₃, based on a 100 mol % total of the composition.

### Aluminoborosilicate Glasses

In embodiments, the disclosed aluminoborosilicate glass composition can comprise a source of, for example:

50 to 60% (B₂O₃+SiO₂),

0.1 to 25% SiO₂,

25% to 59.9% B₂O₃,

2 to 10% Al₂O₃,

1 to 3% P₂O₅,

4 to 10% Na₂O,

6 to 10% K₂O,

6 to 10% MgO, and

20 to 30% CaO, based on a 100 mol % total of the composition.

In embodiments, the aluminoborosilicate glass composition can further comprise a source of SO₃ from 0.2 to 2 mol % SO₃, based on a 100 mol % total of the composition.

In embodiments, a more preferred glass composition can comprise a source of, for example:

50 to 60% (B₂O₃+SiO₂),

4 to 25% SiO₂,

25% to 56% B₂O₃,

2 to 10% Al₂O₃,

1 to 3% P₂O₅,

4 to 8% Na₂O,

6 to 10% K₂O,

6 to 10% MgO,

20 to 24% CaO, and

0.2 to 2% SO₃, based on a 100 mol % total of the composition.

In embodiments, the disclosed alumino-borate or aluminoborosilicate glass composition can further comprise, for example, a form factor selected from a hollow microsphere, a solid microsphere, or a combination or mixture thereof, that is, where the glass composition has a particle shape, such as a sphere, egg-shape, or like geometry.

In embodiments, the hollow microsphere, a solid microsphere, or a combination thereof, can have, for example, a diameter of from 1 to 1000 microns.

In embodiments, the hollow microsphere can have a density, for example, of from 0.1 to 1.5 g/cm³.

In embodiments, the form factor can have associated therewith, for example, within a hollow microsphere or on the surface of a microsphere, a pharmaceutical, a nutri-ceutical, and like biologically active substances, or a performance or formulation enhancing substance, or a combination thereof.

Representative examples of the disclosed aluminoborate compositions and the disclosed aluminoborosilicate compositions are listed in Table 5. Example C-1 is a comparative example, which is free of Al₂O₃.

In embodiments, the disclosed compositions can be free of or substantially free of at least one of, for example, Fe₂O₃, ZnO, CuO, ZnO, and TiO₂, or any combination or mixtures thereof.

In embodiments, the disclosed glasses can typically be melted at a temperature below 1300° C., and in certain embodiments below 1200° C., making it possible to melt in a relatively small commercial glass tank. Microspheres can be produced using, for example, a flame forming technique with the disclosed compositions. Hollow spheres can be obtained in compositions containing a blowing agent (e.g., SO₃).

In embodiments, the glass compositions can be fashioned into solid microspheres (microscope image not shown).

In embodiments, glass microspheres can be prepared by, for example:

preparing a semiproduct (frit) of at least one of the disclosed compositions, which semi-product consists of powders of defined chemical and granulometric compositions; and

forming glass microspheres from the semi-product.

A sol-gel or a flame forming technique are widely used methods to produce glass microspheres. A sol-gel process generally includes the preparation of an aqueous solution of basic silicate containing additional special reagents (e.g., boric acid, urea, etc.), subsequent drying of the solution in a spray dryer, and fabrication of powders of defined granulometric composition, chemical treatment of the glass microspheres in acid solution to remove excess basic components, washing in water, and drying of the finished products (see V. V. Budov, supra.). In a flame forming process, glass microspheres are fabricated from previously synthesized glass powders. The glass frits are spheroidized by, for example, passing the frits through a flame of a gas-oxygen burner at a temperature of from 1000 to 1800° C., or through a vertical split furnace having a similar temperature range. The diameter of the spheres can be, for example, from 1 to 1000 microns, depending on the size of the glass frits. Fine frits (e.g., less than 100 microns) can be obtained using, for example, a jet mill, an attrition mill or ball mill; coarse particles can be produced by crushing glass using, for example, a steel mortar and pestle.

In embodiments, hollow glass microspheres can be produced by adding a blowing agent such as SO₃ into the glass batch compositions (microtomed microscopic image obtained but not shown). The blowing agent decomposes and releases gas to form a single hollow cavity at the center of the sphere during the spheroidizing process. The density of the hollow spheres can be determined by the concentration of the blowing agent included in the glass, and can vary, for example, from 0.1 to 1.5 g/cm³. The hollow glass microspheres exhibit substantial advantages over plastics microbeads, for example: they are more environmentally friendly and more biodegradable; they can provide additional functions or benefits such as wound healing, and anti-oxidation; they have a higher refractive index, making them appealing for cosmetic and beauty enhancement products; they can produce a luxurious or lubricious tactile sensation on the skin; they can have a good oil absorption rate and can improve the compatibility between different ingredients; and the glass compositions can be easily modified to incorporate desired functions.

Methods for making glass microspheres are known, see for example, U.S. Pat. Nos. 3,323,888, and 6,514,892. Methods for making hollow glass microspheres include, for example, U.S. Pat. Nos: 2,978,339; 3,323,888; 5,14,892; and 6,254,981; and Campbell, J. H., et al., Preparation and Properties of Hollow Glass Microspheres for Use in Laser Fusion Experiments,” Technical Report No. UCRL-53516, Lawrence Livermore National Lab., CA (USA), Nov. 1, 1983.

### EXAMPLES

The following Examples demonstrate making, use, and analysis of the disclosed microbead compositions and methods of use in accordance with the above general procedures.

### Example 1

Preparation of 2 wt % PGA and CaCO₃ composite microbeads having 10 wt % calcium carbonate. Composite microbeads were prepared as follow: First, a 2 wt % solution of polygalacturonic acid (PGA) was prepared by dissolving polygalacturonic acid sodium salt , (Sigma #P3850), into ultra-pure (UP) water at 90° C. for 2 hrs with stirring. To this solution was added 0.7 micrometer particles of precipitated calcium carbonate CaCO₃, Albafil PCC available from Mineral Technologies, Inc, to achieve a 10% wt CaCO₃ suspension.

The CaCO₃ and PGA suspension was filtered using a 20 micron polypropylene (PP) filter under vacuum to eliminate coarse particles. Four hundred milliliters of a 10% w/v of calcium chloride in a water/ethanol (75/25:v/v), solution, was added to a beaker and stirred slowly using a magnetic stirrer as a gelling bath.

Droplets were produced by running 25 mL of the CaCO₃ and PGA suspension into the gelling bath using a syringe equipped with a 30 Gauge needle and applying a pressure of about 2 bars. Beads were further crosslinked in the calcium chloride bath for several minutes before being washed four times with UP water.

The composite microbeads were then collected by filtration and dried in an oven at 50° C. The obtained beads were highly opaque and had a narrow particle size distribution

Dried composite particles prepared according to above. These composite beads were prepared from a polygalacturonic acid hydrogel containing 10 wt % CaCO₃ as an abrasive filler. A wrinkled appearance aspect results from shrinkage of the PGA hydrogel upon drying. The particles have an average size of about 0.8 to 1 mm. FIG. 4 shows exemplary composite microbeads after drying (i.e., PGA bound to a CaCO₃ core particle). FIG. 5 shows exemplary composite microbeads after rehydration (i.e., PGA bound to a CaCO₃ core particle).

After rehydration in water the beads show a smoother surface but the size of the particle did not change significantly. The PGA shell looks soft and glossy. The microbeads prepared from a hydrogel containing 0 wt % CaCO₃ abrasive filler, only poorly rehydrate and their uniform shape is maintained.

### Example 2

Core-shell microbead formation procedure 5 mL of a 10% (v/v) acetic acid was added to 200 mL of 4% aqueous solution of PGA (with or without pigment) in a 500 mL round bottomed flask with 4 necks, fitted with an overhead mechanical above and a magnetic stirrer below. The center neck of the round bottomed flask was fitted with a mechanical stirrer shaft having a stirrer paddle and an overhead mechanical stirrer set to 250 to 300 rpm. In addition, a magnetic stirrer bar was added to the solution and the magnetic stirrer set to 500 to 600 rpm. One of the necks of the flask was fitted with a gas inlet adaptor which is supplied with a low flow of nitrogen with a gas bubbler attachment to vent excess pressure even though the reaction is not oxygen sensitive.

1.0 g of 50 mesh CaCO₃ was added slowly (e.g., by tapping CaCO₃ on weighing paper, or using a sieve over about 2 to 3 min) to the PGA solution while stirring and allowed to react an additional 30 min. The product beads were isolated by filtration through a polyester mesh and washed 2×250 mL of DI water, and then 2×100 mL EtOH. The beads were then transferred to an evaporating dish and soaked in 200 mL of 5% solution of CaCl₂ for 60 minutes. The solution was decanted and the resulting beads washed with DI water (2×50 mL), and then soaked in EtOH for 30 min to prevent agglomeration. The solvent was decanted and the resulting beads were dried in an oven set to 65° C. for 30 minutes, then 100° C. for an additional 30 minutes.

### Example 3

Alternative core-shell microbead formation and post processing—silica core and alginate shell 5 mL of a 10% (v/v) acetic acid was added to 200 mL of 4 wt % aqueous solution of alginate (Protanal LF from FMC) in a 500 mL round bottomed flask outfitted and operated as in Example 1. 1.0 g of 50 mesh silica gel (pre-soaked in a 5 wt % aqueous solution of CaCl₂ overnight, then washed, and dried) was added slowly to the PGA solution while stirring and allowed to react for 30 min. The product beads were isolated and washed as in Example 1. The beads were then transferred to an evaporating dish and soaked (60 min) in 100 mL of a 10 wt % solution of tetraethyl orthosilicate (TEOS) in ethanol as an additional crosslinker. The ethanol was decanted and 200 mL of 5% solution of CaCl₂ (in DI water) was added, and the beads soaked for 60 minutes. The solution was decanted and the resulting beads washed with DI water (2×50 mL), and then soaked in EtOH for 30 min. The solvent was decanted and the resulting beads were dried in an oven set to 65° C. for 30 minutes, then 100° C. for an additional 60 minutes.

### Example 4

Exfoliation Formulation Beads produced using the methods described in Examples 1 and 2 were added to a carrier liquid made from a commercial exfoliation soap by removing the commercial plastic microbeads by filtration (Neutrogena Oil-Free Acne Wash: Pink Grapefruit foaming scrub or Irish Spring® Deep Action Scrub, to represent exfoliant formulations having respective pH values of 3.5 and pH 6), or to a 0.25 wt % solution of Carbopol® 980 (Carbopol® Rheology Modifiers available from Lubrizol) in water with pH adjusted with 0.5 N NaOH to pH 7 in a bead:carrier suspension w/w ratios of 1:99 (commercial soap) and 5:95 (Carbopol solution). The suspension was thoroughly mixed to ensure a homogeneous dispersion of the microbeads in the carrier.

Formulation Evaluation Coating durability (also referred to as Crock Resistance) refers to the ability of a surface coating to withstand repeated rubbing with a cloth. The Crock Resistance test is meant to mimic the physical contact between garments or fabrics with a touch screen device and to determine the durability of the coatings disposed on the substrate after such treatment. The Crock Resistance test was modified in the present disclosure to evaluate the relative abrasiveness of exfoliant formulations with respect to controls and commercially available exfoliant products.

A Crockmeter is a standard instrument that is used to determine the Crock resistance of a surface subjected to such rubbing. The Crockmeter subjects a glass slide to direct contact with a rubbing tip or “finger” mounted on the end of a weighted arm. The standard finger supplied with the Crockmeter is a 15 mm diameter solid acrylic rod. A clean piece of standard crocking cloth is mounted to this acrylic finger. The finger then rests on the sample with a standard pressure of 900 g and the arm is mechanically moved back and forth repeatedly across the sample in an attempt to observe a change in the durability/crock resistance. The Crockmeter used in the tests described herein is a motorized model that provides a uniform stroke rate of 60 revolutions per minute. The Crockmeter test is described in ASTM test procedure F1319-94, entitled “Standard Test Method for Determination of Abrasion and Smudge Resistance of Images Produced from Business Copy Products,” the contents of which are incorporated herein by reference in their entirety.

Crock resistance or durability of the test image target labels with respect to a test formulation is determined by a trained human evaluator with respect to the comparative standard formulations after a specified number of wipes. A “wipe” is defined as two strokes or one cycle, of the nitrile rubber modified rubbing tip or finger.

An electronic Crockmeter with an acrylic Crock finger (15 mm diameter) with felt (supplied with Crockmeter) at the contact tip end of the Crock finger was modified by fitting the contact tip end with a nitrile rubber cap. The test substrate was a 1×3 microscope slide (0.7 mm thickness) with a weather proof label (Avery® White WeatherProof™ Laser Mailing Labels, 5520) printed with a test pattern and a target circle for dispensing soap with microbeads. The Crock finger was wiped with water to remove soap and microbead residue and dried prior to each run. The path length of the Crock finger was a 50 mm straight line and the constant force applied was 9 N (65.1 pound foot per second squared) and not a standard pressure of 900 g. One cycle consists of a total of 100 mm travel distance from a starting point at one end of a 50 mm travel path to the other end and then back to the start.

0.07 g to 0.10 g of a formulation sample of a soap having microbeads was dispensed at the starting point (i.e., the printed circle where the Crock finger is first placed on the sample) using a microspatula to spread the sample to cover the entire circle area. The Crock finger was placed directly onto the surface bounded by the circle then setting the Crockmeter to 50 cycles, and pressing start to initiate the test. After the test was complete, the finger was lifted, and the test sample was removed for imaging. Relative abrasion or wear was determined by scratches, removal, smearing, or a combination of these Crock finger artifacts on the printed test pattern. Table 1 provides a summary of relative abrasion or wear and their respective ratings. Table 2 provides a summary of relative abrasion or wear for commercial exfoliation formulation controls. Table 3 provides a summary of relative abrasion or wear for evaluated disclosed exfoliation formulations. Table 4 provides a listing of elastic modulus literature values for selected comparative and experimental material components.

The resultant test pattern was used to rate the exfoliation formulation by visual inspection using a rating scale of 1 to 5, from no abrasion to increasingly harsher abrasion. Each rating is also defined by a reference material which generates the abrasion rating after testing as described above. The respective ratings, numbers from 1 to 5, were characterized according to the following criteria and typical formula results:
1. No change to the printed test pattern. An Irish Spring soap formulation having its microbead content removed provides this 1 rating.
2. Minor wear to test pattern with soap picking up some color from the test pattern but no scratches along abrasion path. When 5 wt % polyethylene microbeads, having an irregular shape, and particle size 200 to 500 micrometers, was added to an Irish Spring soap sample this evaluated formula produced a 2 rating.
3. Clear and visible wear to test pattern in both black ink and colored squares with soap picking up color from the test pattern but no scratches along abrasion path. When 5 wt % of 45 micron hollow soda lime silicate glass bubbles were added to Irish Spring soap this evaluated formula produced a 3 rating.
4. Significant wear to the test pattern in both black ink and colored squares with soap picking up color from the test pattern and light scratches along abrasion path. When 5 wt % of spherical silica gel (75 to 200 microns) was added to a Irish Spring soap this evaluated formula produced a 4 rating.
5. Significant wear and clearly visible scratches (i.e., white lines along the Crock finger travel path in colored patches). When a 5 wt % of irregular shaped calcium carbonate (about 850 microns) was added to a Irish Spring soap this evaluated formula produced a 5 rating.

TABLE 1
Rating Scale for Relative Abrasion or Wear.

| | Reference | |
|---|---|---|
| | particle | Remarks^{1.} (visual assessment of |
| Rating | material | reference image) |
| 1 | soap only | no changes |
| 2 | polyethylene, | soap picks up some color from ink but |
| | irregular | otherwise no visible scratches along |
| | | abrasion path, slight wear |
| 3 | glass bubble, | Soap picks up color from ink, visual |
| | spherical | defects in black ink, clear wear on black |
| | | ink and colored squares |
| 4 | spherical silica | Clear, significant wear on black lines and |
| | gel | colored squares, light scratches along path |
| | | of abrasion |
| 5 | sand, CaCO₃ | Clear wear on black and colored squares, |
| | irregularly | clear and visible scratches along |
| | shaped | path of abrasion |

^{1.}Reference image not shown but available.

TABLE 2
Summary of Relative Abrasion or Wear for Commercial Exfoliation
Formulation Controls.

| | Commercial | | microbead size | shape (core |
|---|---|---|---|---|
| | Product | ranking | (microns) | material) |
| carrier | Irish Spring ® Body | 1 | N/A | N/A |
| controls | wash Deep Action Scrub - | | | |
| (bead-free) | beads removed | | | |
| | Neutrogena - beads | 1 | N/A | N/A |
| | removed | | | |
| commercial | Irish Spring ® Body | 1.5 | 500 | spherical, hydrogenated |
| products | wash Deep Action Scrub | | | Castor oil and irregular, |
| (as-is) | | | | apricot seed powder |
| | Neutrogena Pink | 2 | 200-500 | irregular, polyethylene |
| | grapefruit | | | (PE) |
| | Olay Daily exfoliating | 2.5 | 500-2000 | irregular, oxidized |
| | with sea salts | | | polyethylene (OPE) |
| | Softsoap Body Scrub | 3 | 200-1000 | irregular, oxidized |
| | Coconut Butter | | | polyethylene (OPE) |

TABLE 3
Summary of Relative Abrasion or Wear for Evaluated
Disclosed Exfoliation Formulations.

| | | | | shell wt % |
|---|---|---|---|---|
| | | | | of total |
| | Bead/particle | | Core size | (dry wt |
| Example | Description | Ranking | (microns) | shell/total) |
| spherical | CaCO₃ spherical | 3 | 125 | — |
| CaCO₃ core | CaCO₃ spherical + | 2.5 | 125 | 20-25% |
| (5 wt %) | shell | | | |
| | CaCO₃ spherical + | 2-2.5 | 125 | 20-25% |
| | shell + pigment | | | |
| spherical glass | spherical glass' | 4-4.5 | 125 | — |
| core (5 wt %) | spherical glass¹ + | 2 | 125 | 40-45% |
| | shell | | | |
| irregular | crystalline CaCO₃, | 5 | 850 | |
| CaCO₃ core (5 | irregular | | | |
| wt %) | (850 microns) | | | |
| | crystalline CaCO₃ | 2-2.5 | 850 | |
| | irregular | | | |
| | (850 microns) + | | | |
| | shell | | | |

^{1.}Aluminoborate composition 2 in Table 5.

TABLE 4
Elastic modulus literature values for selected comparative and
experimental material components.

| Material | Elastic modulus (GPa) |
|---|---|
| glass | 50-90 |
| polyethylene | 0.1-0.8 |
| Nylon, acrylics | 2 to 4 |
| CaCO₃ calcite | 70-90 |
| hydrogel modulus (general) | 10 kPa to 500 kPa |

### Example 5

Method Of Making An Aluminoborate Glass Aluminoborate example composition 2 listed in Table 5 was melted in an electric furnace using a batched source materials including boric acid, alumina, sodium carbonate, potassium carbonate, limestone, magnesia, calcium phosphate, and sodium sulfate. Prior to melting, the batches were vigorously mixed in a plastic jar using a Turbula® mixer. Then they were transferred to a platinum crucible with an internal volume of approximately 650 cc. The crucible was then loaded into an annealing furnace to calcine the batch at 250° C. for 24 hr. The calcined bathes were melted at 1200° C. for 6 hr and then glass melt was poured on a steel plate, annealed at 500° C.

Representative examples of the disclosed aluminoborate compositions that can be used to prepare microbead glass cores are listed in Table 5 as Examples 2 to 10. Representative examples of the disclosed aluminoborosilicate compositions that can be used to prepare microbead glass cores are listed in Table 5 as Examples 11 to 15. A comparative borate composition that is free of alumina is composition C-1. The aforementioned aluminoborate compositions and aluminoborosilicate compositions are disclosed in the above mentioned copending patent application ______ (not yet assigned)(SP16-134PZ).

TABLE 5.
Listing of disclosed example aluminoborate compositions (Ex. 2 to 10) and aluminoborosilicate
compositions (Ex. 11 to 15) suitable for microbead glass cores.

| Oxides | | | | | | | | |
|---|---|---|---|---|---|---|---|---|
| (mol %) | C-1^{1.} | 2 | 3 | 4 | 5 | 6 | 7 | 8 |
| B₂O₃ | 54.6 | 50.5 | 48.8 | 38.6 | 34.6 | 50.4 | 50.3 | 50.1 |
| SiO₂ | 0.0 | 0.0 | 0.0 | 0.0 | 0.0 | 0.0 | 0.0 | 0.0 |
| A1₂O₃ | 0.0 | 7.4 | 10.7 | 16.0 | 20.0 | 7.4 | 7.4 | 7.3 |
| P₂O₅ | 1.7 | 1.6 | 1.5 | 1.7 | 1.7 | 1.6 | 1.6 | 1.6 |
| Na₂O | 6.0 | 5.6 | 5.4 | 6.0 | 6.0 | 5.5 | 5.5 | 5.5 |
| K₂O | 7.9 | 7.3 | 7.1 | 7.9 | 7.9 | 7.3 | 7.3 | 7.2 |
| MgO | 7.7 | 7.1 | 6.9 | 7.7 | 7.7 | 7.1 | 7.1 | 7.1 |
| CaO | 22.1 | 20.5 | 19.7 | 22.1 | 22.1 | 20.4 | 20.4 | 20.3 |
| SO₃ | 0.0 | 0.0 | 0.0 | 0.0 | 0.0 | 0.2 | 0.5 | 0.9 |
| Oxides | | | | | | | | |
| (mol %) | 9 | 10 | 11 | 12 | 13 | 14 | 15 | |
| B₂O₃ | 49.6 | 50.3 | 45.7 | 41.1 | 36.6 | 32.0 | 27.4 | |
| SiO₂ | 0.0 | 0.0 | 4.6 | 9.1 | 13.7 | 18.3 | 22.9 | |
| A1₂O₃ | 7.3 | 7.3 | 7.3 | 7.3 | 7.3 | 7.3 | 7.3 | |
| P₂O₅ | 1.5 | 1.6 | 1.6 | 1.6 | 1.6 | 1.6 | 1.6 | |
| Na₂O | 5.5 | 5.5 | 5.5 | 5.5 | 5.5 | 5.5 | 5.5 | |
| K₂O | 7.2 | 7.2 | 7.2 | 7.2 | 7.2 | 7.2 | 7.2 | |
| MgO | 7.0 | 7.0 | 7.0 | 7.0 | 7.0 | 7.0 | 7.0 | |
| CaO | 20.1 | 20.2 | 20.2 | 20.2 | 20.2 | 20.2 | 20.2 | |
| SO₃ | 1.8 | 0.9 | 0.9 | 0.9 | 0.9 | 0.9 | 0.9 | |

^{1.}Example C-1 is a comparative example.

### Example 6

METHOD OF MAKING A SOLID MICROSPHERE ALUMINOBORATE GLASS WITH THE GLASS OF EXAMPLE 1 Solid glass microspheres can be prepared from any of the disclosed example glass source material composition(s) of Table 5. The production of glass microspheres generally includes two stages: preparation of semi-product (frit), which consists of powders of defined chemical and granulometric compositions; and forming of glass microspheres. Sol-gel and flame-forming are the two most widely used methods to produce glass microspheres. In the flame forming process, glass cullets of the approximate desired particle sizes are first prepared by crushing the glass using a steel mortar and a pestle, then milling with, for example, jet milling, attrition milling, ball milling, or like methods. Additionally or alternatively, the crushed or milled particles can be spheroidized by passing the reduced particles through a flame of a gas-oxygen burner at a temperature of from 1000 to 1800° C., or through a vertical split furnace at a similar temperature. The diameter of the glass microspheres can be, for example, from 1 to 1000 micrometers, depending on the size of the glass frits. A specific composition used for making solid microbeads was composition 2 in Table 5.

### Example 7

METHOD OF MAKING A HOLLOW MICROSPHERE ALUMINOBORATE GLASS WITH THE GLASS OF EXAMPLE 1 Hollow glass microspheres are produced by batching a blowing agent such as SO₃ into the glass compositions. The blowing agent thermally decomposes and releases gas to form a single hollow cavity at the center of the sphere during the spheroidizing process. Similar to the solid glass microspheres, the production of hollow glass microspheres also includes two stages: preparation of semi-product (frit), which consists of powders of defined chemical and granulometric compositions; and forming the glass microspheres. Glass cullet of desired particle sizes are first prepared by crushing glass using steel mortar and pestle, then milling, for example, jet milling, attrition milling, or ball milling. The milled particles can be spheroidized by passing the particles through a flame of gas-oxygen burner at a temperature of from 1000 to 1800° C. or through a vertical split furnace of a similar temperature range. The diameter of the glass microspheres can be from 1 to 1000 micrometers, depending on the size of the glass frits. A specific composition used for making hollow microbeads was composition 2 in Table 5.

### Comparative Example 8

Exfoliation evaluation Comparative exfoliation suspension formulations were prepared with different beads selected from: polyethylene microbeads (200-500 microns) that were isolated from Neutrogena Oil-Free Acne wash; CaCO₃ (50 mesh, irregular shaped; core particles without a hydrogel shell); CaCO₃ (125 micrometers, spherical; i.e., core particles without a hydrogel shell); spherical silica gel (Sigma-Aldrich, 75 to 200 microns); silica gel (Sigma-Aldrich, 200 to 500 microns); and sand (Fisher Scientific, sea sand, 20 to 30 mesh; S25-500), and the same commercial exfoliation soap (i.e., commercial carrier with the microbeads removed by filtration). Abrasive particles were prepared with 1 g of 50 mesh CaCO₃ being added to form a 1:99 and a 5:95 w/w ratio (bead:carrier) suspension formulation, and the resulting formulation was mixed thoroughly to ensure homogeneous dispersion of microbeads in the carrier.

The abrasion/wear characteristics of the resultant formulations were tested using an electronic Crockmeter as described in Example 3.

Commercially available exfoliation formulations listed in Table 6 were likewise dispensed, and used as-is, i.e., no microbeads added or removed, and tested using the Crockmeter test and procedure described above. The abrasion ranking or rating for suitable exfoliant formulations was from 1.5 to 3. The abrasion ranking or rating for unsuitable exfoliant formulations was less than 1.5 (i.e., insufficient exfoliation) and greater than 3 (i.e., excess exfoliation or abrasion).

TABLE 6
Characterization and evaluation of selected comparative
exfoliation formulas.

| Comparative | Bead | | core size | |
|---|---|---|---|---|
| Examples 8 | description | ranking | (microns) | core shape |
| comparative | Polyethylene | 2 | 200-500 | irregular |
| microbeads (5 | (PE) Beads from | | | |
| wt %) | Neutrogena | | | |
| | 3M glass bubbles | 3 | ca. 45 | spherical |
| | K37 soda lime | | | |
| | silicate | | | |
| | porous silica gel | 5 | 200-500 | irregular |
| | (Sigma) | | | |
| | porous silica gel | 4 | 75-200 | spherical |
| | (Sigma) | | | |
| | sand | 5 | 600-850 | irregular |

### Example 9

Accelerated durability testing as a measure of formulation shelf-life or shelf-stability A durability test for the core material alone and the core-shell microbeads was conducted at room temperature using a pH 4 acetate buffer solution prepared by mixing aqueous 0.1 M acetic acid (1694 mL) and aqueous 0.1 M sodium acetate (306 mL). CaCO₃ (850 micron particle size, 7.7 g) was added to the buffered solution (200 mL) to achieve a surface area-to-volume (SAN) ratio of 0.2 cm⁻¹. After addition, the solids were allowed to stand for 24 hr without agitation to mimic microbeads in a formulation. The microbeads were separated out by filtration and the leachates were tested by ICP-MS for Ca²⁺ ion concentration. The Ca²⁺ leach rate was calculated by micro-g/cm²/day. The same test can be done using a pH 6 buffer to measure the effects of higher pH on leach rate. The results show that in all cases (CaCO₃ and the glass tested) the leach rate was higher in a lower pH solution.

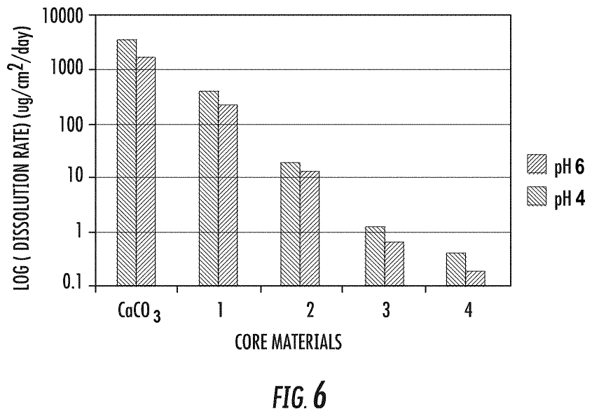

FIG. 6 shows accelerated dissolution kinetics of Ca²⁺ ion release from exemplary core materials (CaCO₃ particles, and glass microbead compositions 1, 2, and 3) as a function of pH 4 (left bar) and pH 6 (right bar) after 24 hrs in acetate buffer. Table 7 lists core compositions that were evaluated by dissolution tests.

TABLE 7
Core compositions tested in dissolution.

| Core | | | | | | | | |
|---|---|---|---|---|---|---|---|---|
| compositions | | | | | | | | |
| (mol %) | CaO | Al₂O₃ | SiO₂ | B₂O₃ | P₂O₅ | Na₂O | K₂O | MgO |
| CaCO₃ | 100 | — | — | — | — | — | — | — |
| particles | | | | | | | | |
| glass | 20.5 | 7.4 | — | 50.6 | 1.6 | 5.6 | 7.3 | 7.1 |
| microbead | | | | | | | | |
| composition 1 | | | | | | | | |
| glass | 49.2 | 9.6 | 40.7 | — | — | — | — | — |
| microbead | | | | | | | | |
| composition 2 | | | | | | | | |
| glass | 20.1 | 14.7 | 65 | — | — | — | — | — |
| microbead | | | | | | | | |
| composition 3 | | | | | | | | |
| glass | 10 | 5 | 84.9 | — | — | — | — | — |
| microbead | | | | | | | | |
| composition 3 | | | | | | | | |

The test can be accelerated by agitation using a magnetic stirrer bar. The same test comparing CaCO₃ dissolution with and without a PGA shell in a pH 4 buffer as described above showed calcium ion concentrations of 466 ppm in the absence of the hydrogel shell, and 252 ppm with the hydrogel shell. The result demonstrated that the shell slows the dissolution of the CaCO₃ core material by approximately half after 24 hrs.

In addition, the relative leach rate of calcium carbonate (with and without hydrogel shell) was measured by the change in the pH buffered solutions as a function of neutralization of the acidic buffer upon dissolution of the calcium carbonate core. In a 20 mL scintillation vial, 10 mL of a 0.1M pH 4 acetate buffer and CaCO₃ (0.39 g) particles were added. In a separate scintillation vial 0.73 g of a core-shell microbead (made of 0.39 g CaCO₃ particles , 0.34 g shell of crosslinked PGA) was added to 10 mL of the pH 4 acetate buffer. 4 drops of a universal pH indicator solution (for pH 4-10, Sigma-Aldrich catalog #36828) and magnetic stirrer bars were added to both vials, the vials were capped and stirred at about 250 rpm. The CaCO₃ core only sample increased in pH to about 5 to 5.5 after 3 hrs and reached pH 7 after 24 hrs (and remained at pH 7 after several more days) while the core-shell microbeads having the hydrogel shell remained at pH 4 after 3 hrs and rose to pH 5.5 after 24 hrs (and remained at pH 5.5 for several more days). This result demonstrates that the hydrogel shell slows down the dissolution (or leach) rate of the calcium carbonate core particle(s).

### Example 11

METHOD OF MAKING A CORE-SHELL MICROBEAD HAVING AN ALUMINOBORATE GLASS CORE Example 2 was repeated with the exception that: a solid microsphere aluminoborate glass of Example 6 was selected as the microbead core in place of the precipitated calcium carbonate CaCO₃ microbead core; the reaction time was 90 mins instead of 30 min; and the aluminoborate glass was presoaked in aqueous 5% CaCl₂ for about 20 hours prior to contacting with the 4% aqueous solution of PGA and acetic acid solution. The isolated core-shell microbeads having a glass core had an overall bead diameter of about 600 to 1,200 microns and a core diameter of about 200 to 400 microns, see FIG. 7.

### Example 12

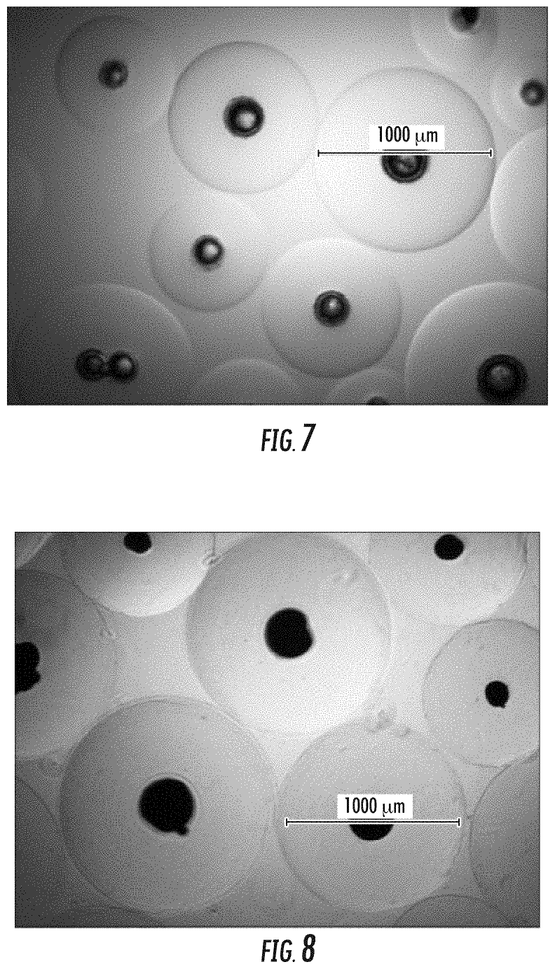

METHOD OF MAKING A CORE-SHELL MICROBEAD HAVING A PRECIPITATED CALCIUM CARBONATE MICROBEAD CORE Example 2 was repeated with the exception that: the PGA solution was mixed with the precipitated calcium carbonate CaCO₃ microbead core particles and stirred for about 20 hrs; the acetic acid was added to the 16 hrs stirred mixture of the PGA solution and the CaCO₃ microbead core particles; and the reaction time was 15 mins instead of 30 min. The isolated core-shell microbeads having calcium carbonate CaCO₃ core particles had an overall bead diameter of about 600 to 1,200 microns and a core diameter of about 200 to 400 microns, see FIG. 8. The microbeads of this Example 12 had a strong resemblance in dimensions and size uniformity to glass core microbeads of this Example 11.

### Example 13

Biodegradability Testing with E. coli. Core-shell beads included: 850 micron CaCO3 with 6% PGA; 850 micron CaCO₃ with 4% PGA; and 150 to 300 micron microbead glass composition 1 with 4% PGA. The beads were incubated in trypticase soy broth with E. coli for 20 hrs and compared with a control (images available but not provided).

The disclosure has been described with reference to various specific embodiments and techniques. However, it should be understood that many variations and modifications are possible while remaining within the scope of the disclosure.

What is claimed is:

1. An exfoliant composition comprising:
a microbead comprising a core and a shell:
the core comprising an abrasive particle having an average particle size of from 50 to 1,000 microns; and
the shell comprising a hydrogel.

2. The composition of claim 1 wherein the abrasive particle is selected from at least one of: CaCO₃, a calcium mineral, a glass, a calcium containing glass, a silica, a porous silica doped with calcium, a sand, or a mixture thereof.

3. The composition of claim 1 wherein the hydrogel is selected from at least one of: a cross-linked polygalacturonic acid; a cross-linked alginate; or a mixture thereof.

4. The composition of claim 3 wherein the cross-linked polygalacturonic acid is cross-linked with a polyvalent cation.

5. The composition of claim 4 wherein the polyvalent cation is selected from at least one of: Ca²⁺, Cu²⁺, Al³⁺, Fe³⁺, Mg²⁺, Pb²⁺, Cd²⁺, Ba²⁺, Sr²⁺, Co²⁺, Ni²⁺, Zn²⁺, Mn²⁺, chitosan, or mixtures thereof.

6. The composition of claim 1 wherein the shell modulates the abrasiveness of the abrasive particle.

7. The composition of claim 3 wherein the type and extent of the crosslinking of the cross-linked polygalacturonic acid controls the environmental biodegradability of the microbead.

8. The composition of claim 1 wherein the shell is durable with respect abrasion, dissolution, or both, and the shell resists acid or base degradation.

9. The composition of claim 1 wherein the microbead has an environmental biodegradability of from 1 week to 5 years.

10. The composition of claim 1 wherein the microbead has a high elastic modulus core of from 50 to 90 GPa, and a low elastic modulus shell of from 100 to 500 kPa.

11. The composition of claim 1 further comprising a carrier.

12. The composition of claim 11 wherein the carrier is selected from a liquid, a soap, a soap solution, a gel, a cream, a lotion, a powder, or a mixture thereof.

13. A method of making the exfoliant composition of claim 1 comprising:
forming a hydrophilic shell on at least a portion of the surface of an abrasive core particle to form a microbead.

14. The method of claim 13 further comprising, sequentially or simultaneously, cross-linking the hydrophilic shell on at least a portion of the surface of the abrasive core particle.

15. The method of claim 14 wherein the cross-linking is accomplished with a polyvalent cation and an acid catalyst.

16. A method of using the exfoliant composition of claim 1 comprising:
contacting a skin surface with the exfoliant composition.

17. The method of claim 16 further comprising mechanically working the exfoliant composition onto or into the skin surface.

18. The method of claim 17 further comprising removing the mechanically worked exfoliant composition from the skin surface.
